(19) United States
(12) Patent Application Publication
(10) Pub. No.: US 2008/0190428 A1
(43) Pub. Date: Aug. 14, 2008

(54) METHOD FOR CONTROLLING CONTINUOUS POSITIVE AIRWAY PRESSURE

(75) Inventor: Ching-Liang YU, Tu-Cheng City (TW)

Correspondence Address: PATENTTM.US, P. O. BOX 82788, PORTLAND, OR, 97282-0788, US

(73) Assignee: APEX MEDICAL CORP., Tu-Cheng City (TW)

(21) Appl. No.: 11/673,854

(22) Filed: Feb. 12, 2007

(51) Int. Cl. A61M 16/00

(52) U.S. Cl. 128/204.23

(57) ABSTRACT

A method for controlling continuous positive airway pressure includes the steps of building up air flow pressure to an initial air flow pressure, increasing the initial air flow pressure to a detection air flow pressure, detecting consistency between the initial air flow pressure and the detection air flow pressure, checking to see if the initial time frame for consistency detection is due, decreasing air flow pressure from the detection air flow pressure to an intermittent air flow pressure and increasing the intermittent air flow pressure to a treatment air flow pressure.

### BACKGROUND OF THE INVENTION

1. Field of the Invention

The present invention relates to a method for helping patients having sleep apnea to sleep and more particularly to a method for controlling continuous positive airway pressure (CPAP) so as to maintain the airway of the patient open to allow the patient to breathe normally.

2. Description of the Prior Art

Currently, a patient having sleep apnea needs to have a CPAP (continuous positive airway pressure) to help maintain his/her airway open so as to breathe normally. It is because when the patient falls asleep, the airway sometimes may collapse due to the slack airway muscle, which seriously endangers the patient's life. However, when the symptom of having the airway muscle being slack is not that serious, the patient may still snore, which causes problems to the person sleeping nearby. The CPAP is a device to continuously provide positive pressure to the patient's airway to help maintain the airway open such that the patient may breathe normally. Currently, this is the best device to treat patients having sleep apnea and snoring problems.

Because each person has his own sleeping habit and the time for a person to fall asleep varies greatly, the CPAP normally has a knob to allow the patient to adjust the air flow pressure, normally between 4 cm/H₂O˜8 cm/H₂O, to the patient's airway. In order to provide a comfortable breathing environment, the CPAP is able to gradually increase its air flow pressure to the patient's airway such that the patient is able to gradually fall asleep without even noticing that an air flow pressure change is being constantly provided to the airway. As time elapses, the air pressure reaches its predetermined value and the CPAP will automatically stop increasing the air flow pressure and maintain this air flow pressure value throughout the entire treatment period. The time required for the CPAP to start from the initial air pressure to the predetermined treatment air pressure is called “Ramp Time” or “Delay Time”, in which time, the air flow pressure from the CPAP will be gradually increased so as to provide a comfortable environment to help the patient to sleep.

With reference to FIG. 1, it is to be noted that Pi is the initial air pressure, Pt is the predetermined treatment air flow pressure and Tr is the ramp time. From the depiction of FIG. 1, it is noted that initially the CPAP will start providing air flow at Pi. Then gradually the air flow pressure Pi is linearly increased to Pt after Tr. Thereafter, the air flow pressure remains unchanged throughout the entire treatment period. The technique shown in the drawing is linear increment. However, non-linear increment technique, such as parabolic curve, has also been available in the art for years.

However, once the CPAP is activated, the current available technique only allows the CPAP to continuously increase the air flow pressure until the predetermined treatment air flow pressure is reached. That is, after the patient wears the CPAP mask and the CPAP is activated, the CPAP will start providing air flow to the patient regardless of whether the patient is still wearing the mask. In a situation where the patient is suddenly fully awake during sleep and decides to get up for a glass of water, the air blower inside the CPAP will still provide air flow to the mask despite the absence of the patient, which is a waste of energy. Also, when the patient returns to sleep again, the CPAP will continuously provide air flow with treatment pressure. Since the treatment air flow pressure is higher than the air flow pressure required by the patient who is just about to sleep, or returns to sleep from matters other than sleep, the air flow with treatment pressure troubles the patient. Thus the patient will have difficulties falling asleep.

In order to obviate the shortcoming, a new technique is developed and introduced to the market, with which the CPAP will drop the air flow pressure once the mask is detected not worn properly or the mask is set aside completely. The air flow pressure drop allows the patient returning from chores other than sleep to fall asleep easily.

### SUMMARY OF THE INVENTION

The primary objective of the present invention is to provide a method for controlling continuous positive airway pressure so as to provide a comfortable breathing environment to help the patient easily fall asleep.

In order to accomplish the aforementioned objective, the method for controlling continuous positive airway pressure of the present invention includes the following steps:

building up air flow pressure to an initial air flow pressure;

detecting existence of a load within a predetermined time frame;

increasing the initial air flow pressure to a detection air flow pressure;

detecting consistency between the initial air flow pressure and the detection air flow pressure;

checking to see if the initial time frame for consistency detection is due;

decreasing air flow pressure from the detection air flow pressure to an intermittent air flow pressure between the initial air flow pressure and a detection air flow pressure; and

increasing the intermittent air flow pressure to the treatment air flow pressure.

From the aforementioned steps of the present invention, it is to be noted that the existence of air flow pressure drop provides a comfortable breathing environment for the patient such that when the patient is just putting back the mask before the treatment air flow pressure is reached, the air flow pressure is decreased to reduce the difficulties in compliance with the air flow pressure required by the patient.

Another objective of the present invention is that the steps of the method has a consistency detection time frame in which existence of a load is constantly being detected so as to provide signal to a microprocessor to control operation of an air blower.

Still another objective of the present invention is that after the consistency detection time frame is due and the existence of the load is not detected, the microprocessor shut-down the operation of the air blower to place the CPAP in a stand-by mode.

Other objects, advantages and novel features of the invention will become more apparent from the following detailed description when taken in conjunction with the accompanying drawings.

### BRIEF DESCRIPTION OF THE DRAWINGS

FIG. 1 is a schematic chart showing the conventional increase of air flow pressure;

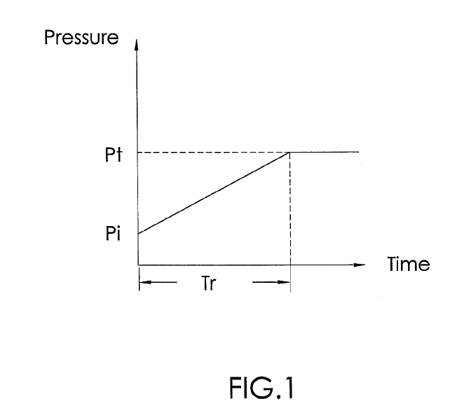

FIG. 2 is a flow chart showing the controlling mechanism of the method of the present invention;

FIG. 3 is a schematic chart showing the increase of air flow pressure of the present invention;

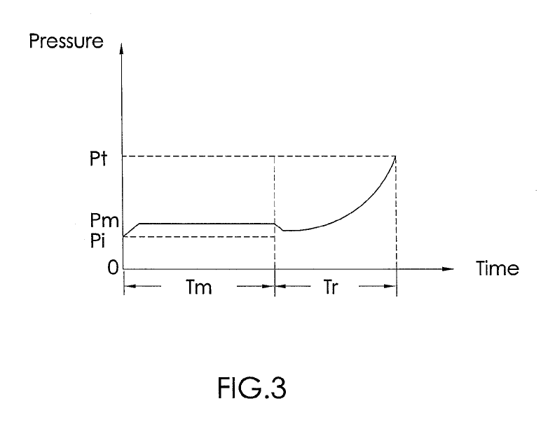

FIG. 4 is a schematic view showing the communication between elements used in the present invention; and

FIG. 5 is a flow chart of the steps of the present invention.

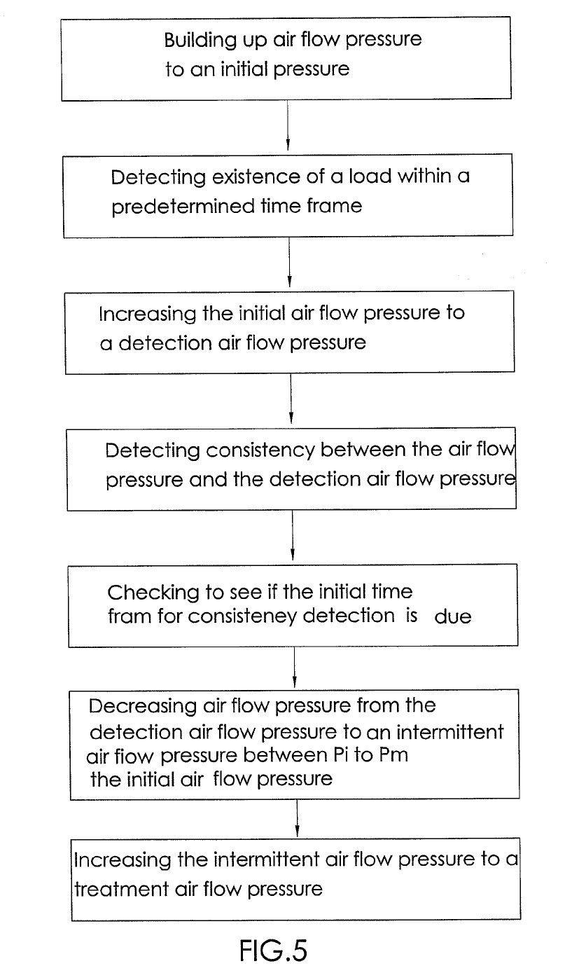

### DETAILED DESCRIPTION OF THE PREFERRED EMBODIMENT

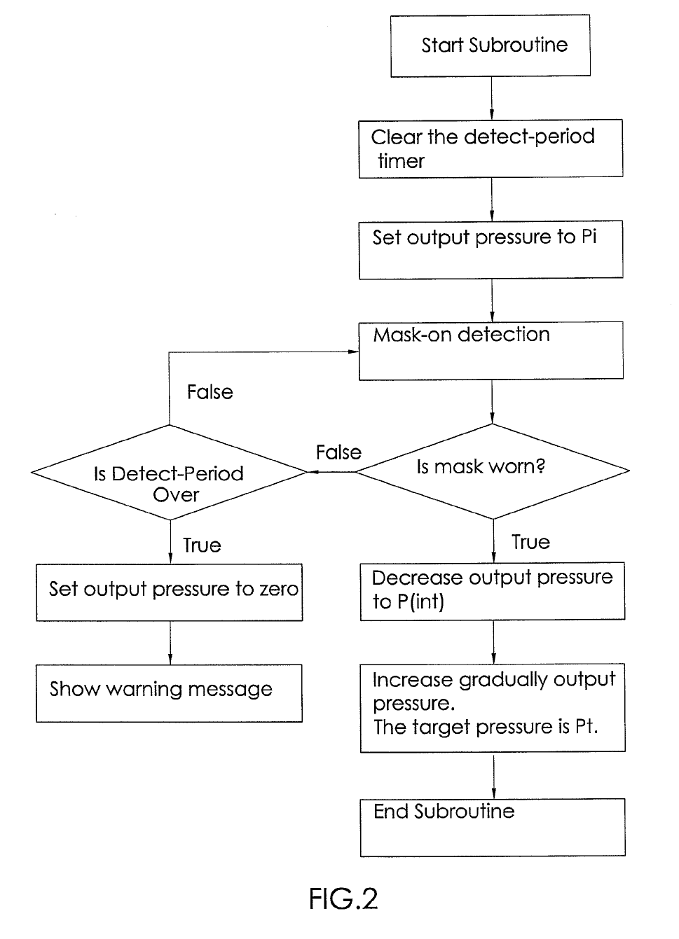

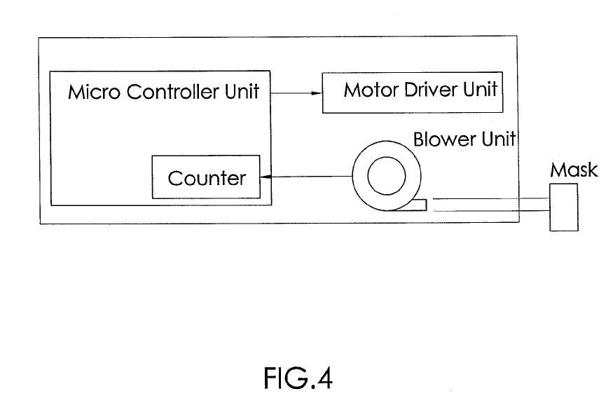

With reference to FIGS. 2 and 4, it is noted that the controlling mechanism of the method of the present invention starts from the initiation of the CPAP. That is, when the power switch of the CPAP is pressed, any record recorded inside a counter inside the CPAP is reset to zero, 0. A treatment air flow pressure of the CPAP is default to be (Pt). After setting the treatment air flow pressure to Pt is finished, the CPAP will immediately start providing an initial air flow pressure (air flow with a pressure value of (Pi)), where Pi<Pt. Within approximately 6 seconds, the initial air flow pressure reaches a detection air flow pressure (Pm). Then a Hall element inside an air blower that is responsible for providing an air flow constantly searches for existence of a load. That is, if the patient properly wears the mask and breathes normally, the air flow coming from the air blower is blocked by the patient's face, which causes a positive pressure. After a period of time, if the Hall element still picks up the existence of the load, the air flow pressure (Pm) from the air blower is dropped to an intermittent air flow pressure which is higher than that of Pi and lower than that of Pm, the detection air flow pressure. In this preferred embodiment, the detection time frame is approximately set to be two (2) minutes. However, the detection time frame presented here is not intended to limit the application of the detection time frame, but only for illustrative purpose. The detection time frame may also be set to any time frame suitable.

After the pressure drop, the CPAP will then gradually increase the air flow pressure from (Pi) to (Pt) and maintain providing the air flow pressure (Pt) throughout the entire treatment time frame, a time in which the patient is asleep.

During the detection time frame, if the Hall element of the air blower does not pick up the existence of the load, the patient is either out of the bed or is not properly wearing the CPAP mask, the Hall element sends a signal to a microprocessor inside the CPAP to suspend the air blower operation, which places the CPAP in a stand-by mode. That is, before the initial detection time frame is due, if the positive pressure from the patient is not picked up by he Hall element of the air blower, the microprocessor stops the air blower operation to shut down entire CPAP operation as a safety measure. On the other hand, once the initial detection time frame is due and after the air flow pressure is drop, the microprocessor, after receiving a signal from the Hall element indicating the existence of the load, speeds-up the air blower operation so as to increase air flow pressure to (Pt).

From the aforementioned description, it is noted that the advantage of the present invention provides an initial detection time frame searching for the existence of a load, a positive pressure against the air flow pressure from the air blower, to be the basis for shutting down the operation of the air blower or to decrease the air flow pressure from (Pm) to somewhere between (Pm) and (Pi). If the positive pressure is not picked up by the Hall element, the CPAP will assume that the patient is not properly wearing the CPAP mask or the patient is not wearing the CPAP mask at all. In either case, if the CPAP continues to send air flow to the CPAP mask, it will be considered as an energy waste. And since there is no one attending the CPAP, potential hazard of causing an electrical short exists.

In summary, it is concluded that the method of the present invention includes the following steps:

building up air flow pressure to an initial air flow pressure;

detecting existence of a load within a predetermined time frame;

increasing the initial air flow pressure to a detection air flow pressure;

detecting consistency between the initial air flow pressure and the detection air flow pressure;

checking to see if the initial time frame for consistency detection is due;

decreasing air flow pressure from the detection air flow pressure to an intermittent air flow pressure between the initial air flow pressure and the detection air flow pressure; and

increasing the intermittent air flow pressure to a treatment air flow pressure.

Furthermore, the load existence detection step may also be applied after the consistency detection step so that after the air flow pressure from the air blower reaches the treatment air flow pressure, the Hall element searches for the existence of the load to see if the CPAP mask is properly worn. After properly wearing the CPAP mask is confirmed, the load existence is confirmed, the step of initial time frame for consistency detection is processed to see whether the air flow pressure drop is to be applied.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the fall extent indicated by the broad general meaning of the terms in which the appended claims are expressed.

What is claimed is:

1. A method for controlling continuous positive airway pressure comprising the steps of:
building up air flow pressure to an initial air flow pressure;
increasing the initial air flow pressure to a detection air flow pressure;
checking to see if the initial time frame for consistency detection is due;
decreasing air flow pressure from the detection air flow pressure to a intermittent air flow pressure between the initial air flow pressure and the detection air flow pressure; and
increasing the intermittent air flow pressure to a treatment air flow pressure.

2. The method as claimed in claim 1 further comprising a step of detecting existence of a load within a predetermined time frame after the air flow pressure building up step and before the initial air flow pressure increasing step.

3. The method as claimed in claim 1 further comprising a step of detecting existence of a load within a predetermined time frame after the consistency detection step and before the checking step.

4. A method for controlling continuous positive airway pressure comprising the steps of:
building up air flow pressure to an initial air flow pressure via an air blower;
detecting existence of a load via a Hall element of the air blower within a predetermined time frame;
increasing the initial air flow pressure by a microprocessor after receiving a signal from the Hall element to a detection air flow pressure;
checking to see if the initial time frame for consistency detection is due;
decreasing air flow pressure from the detection air flow pressure to an intermittent air flow pressure between the initial air flow pressure and the detection air flow pressure; and
increasing the intermittent air flow pressure to a treatment air flow pressure.

5. The method as claimed in claim 4 farther comprising a step of detecting existence of a load within a predetermined time frame via the Hall element after the consistency detection step and before the checking step.

6. The method as claimed in claim 5, wherein after the initial time frame for consistency detection is due and the load is not detected by the Hall element, the microprocessor places the air blower in a stand-by mode.
